(19) United States
(12) Patent Application Publication
(10) Pub. No.: US 2006/0204546 A1
(43) Pub. Date: Sep. 14, 2006

(54) Methods and systems for delivering immunosuppressant and anti-inflammatory agents from a stent

(75) Inventors: Thai Minh Nguyen, Santa Clara, CA (US); John F. Shanley, Redwood City, CA (US); Frank Litvack, Los Angeles, CA (US); Theodore L. Parker, Danville, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/375,454

(22) Filed: Mar. 14, 2006

Related U.S. Application Data
(60) Provisional application No. 60/662,040, filed on Mar. 14, 2005.

(51) Int. Cl. A61F 2/06

(52) U.S. Cl. 424/426; 623/1.11

(57) ABSTRACT

A method for decreasing the level of restenosis following a stent placement medical intervention involves the continuous administration of a dose of an immunosuppressant or anti-inflammatory agent from reservoirs in a stent to vascular tissue in need of treatment in a controlled, two phase drug release profile. It is envisioned that the vascular tissue in need of treatment is arterial tissue, specifically coronary arterial tissue. The agent or drug can be the calcineurin inhibitor Pimecrolimus. The drug can be held within reservoirs in the stent in a drug delivery matrix comprised of the drug and a bioresorbable polymeric material and optionally additives to regulate the drug release.

### CROSS REFERENCE TO RELATED APPLICATIONS

This application claims priority to U.S. Provisional Patent Application Ser. No. 60/662,040, filed Mar. 14, 2005, the entire contents of which are incorporated here by reference.

### BACKGROUND

Most coronary artery-related deaths are caused by atherosclerotic lesions which limit or obstruct coronary blood flow to heart tissue. To address coronary artery disease, doctors often resort to percutaneous transluminal coronary angioplasty (PTCA) or coronary artery bypass graft (CABG). PTCA is a procedure in which a small balloon catheter is passed down a narrowed coronary artery and then expanded to re-open the artery. The major advantage of angioplasty is that patients in which the procedure is successful need not undergo the more invasive surgical procedure of coronary artery bypass graft. A major difficulty with PTCA is the problem of post-angioplasty closure of the vessel, both immediately after PTCA (acute reocclusion) and in the long term (restenosis).

Coronary stents are typically used in combination with PTCA to reduce reocclusion of the artery. Stents are introduced percutaneously, and transported transluminally until positioned at a desired location. These devices are then expanded either mechanically, such as by the expansion of a mandrel or balloon positioned inside the device, or expand themselves by releasing stored energy upon actuation within the body. Once expanded within the lumen, these devices, called stents, become encapsulated within the body tissue and remain a permanent implant.

Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen. Despite the introduction of improved surgical techniques, devices, and pharmaceutical agents, the overall restenosis rate is still reported in the range of 10% to 25% within six to twelve months after an angioplasty procedure for bare metal stents. To treat this condition, additional revascularization procedures are frequently required, thereby increasing trauma and risk to the patient.

While the exact mechanisms of restenosis are still being determined, certain agents have been demonstrated to reduce restenosis in humans. One example of an agent which has been demonstrated to reduce restenosis when delivered from a stent is paclitaxel, an anti-mitotic agent which interferes with smooth muscle cell proliferation. However, many other agents which operate by different mechanisms of action may also be useful to treat restenosis. Other agents which may be useful in treating restenosis are the immunosuppressants and the anti-inflammatories.

### SUMMARY OF THE INVENTION

The present invention relates to a method and system for decreasing restenosis following stenting by administration of an immunosuppressant or anti-inflammatory agent in a controlled drug release profile from a reservoir.

The invention also relates to a system and method for controlled release of an immunosuppressant or anti-inflammatory agent with drug delivery profile programmed to match the timing of the inflammatory response in the body.

In accordance with one aspect of the invention a stent for reducing restenosis is comprised of an expandable stent wherein a matrix is affixed to the stent for delivery of Pimecrolimus to a blood vessel, wherein the bioresorbable matrix includes about 100 μg to about 400 μg of Pimecrolimus normalized for a 3 mm by 16 mm stent.

In accordance with a further aspect of the invention, a method of reducing restenosis comprises the steps of providing a drug delivery stent having a dosage of Pimecrolimus provided in a plurality of reservoirs for delivery to an artery, the dosage arranged such that at least 40% of the Pimecrolimus is released from the stent within 48 hours of implantation of the stent in the artery, implanting the stent within an artery of a patient and delivering Pimecrolimus from the stent to the artery such that substantially all the Pimecrolimus is released from the stent within about 3 weeks.

In accordance with yet a further aspect of the invention, a stent for reducing restenosis is comprised of an expandable stent having a plurality of reservoirs and an anti-inflammatory or immunosuppressive agent provided within the reservoirs in a matrix. The matrix is formulated to release the immunosuppressant agent in a two phase release profile with a first phase releasing at least 50% of the agent within a first 48 hours after stent implantation, and a second phase releasing the remaining agent thereafter at a slower release rate.

In accordance with another aspect of the invention, a stent for reducing restenosis is comprised of an expandable stent having a plurality of reservoirs, and a matrix of Pimecrolimus and bioresorbable polymer provided in the plurality of openings. The matrix is at least 50% Pimecrolimus.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1.

FIG. 3a is a side cross sectional view of an example of an opening in a stent showing a matrix with a cap region, a therapeutic agent region and a barrier region.

FIG. 3b is a graph of the cumulative release of Pimecrolimus from the stent of FIG. 3a.

FIG. 4a is a side cross sectional view of another example of an opening in a stent showing a matrix with a therapeutic agent region and a barrier region.

FIG. 4b is a graph of the cumulative release of Pimecrolimus from the stent of FIG. 4a.

FIG. 5a is a side cross sectional view of an example of an opening in a stent showing a matrix with two therapeutic agent regions with different agent concentrations and a barrier region.

FIG. 5b is a graph of the cumulative release of Pimecrolimus from the stent of FIG. 5a.

FIG. 6 is a graph of the cumulative release of Pimecrolimus from an alternative stent of FIG. 5a.

### DETAILED DESCRIPTION

A method for decreasing the level of restenosis following a stent placement medical intervention involves the continuous administration of a dose of an immunosuppressant or anti-inflammatory agent from reservoirs in a stent to vascular tissue in need of treatment in a controlled, two phase drug release profile. It is envisioned that the vascular tissue in need of treatment is arterial tissue, specifically coronary arterial tissue.

In one example described in detail herein the agent or drug is the calcineurin inhibitor Pimecrolimus. The drug will be held within the reservoirs in the stent in a drug delivery matrix comprised of the drug and a polymeric material and optionally additives to regulate the drug release. Preferably the polymeric material is a bioresorbable polymer.

The following terms, as used herein, shall have the following meanings:

The terms “drug” and “therapeutic agent” are used interchangeably to refer to any therapeutically active substance that is delivered to a living being to produce a desired, usually beneficial, effect.

The term “matrix” or “biocompatible matrix” are used interchangeably to refer to a medium or material that, upon implantation in a subject, does not elicit a detrimental response sufficient to result in the rejection of the matrix. The matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, preferably less than one year, while maintaining any requisite structural integrity in that same time period.

The term “openings” includes both through openings and recesses.

The term “pharmaceutically acceptable” refers to the characteristic of being non-toxic to a host or patient and suitable for maintaining the stability of a therapeutic agent and allowing the delivery of the therapeutic agent to target cells or tissue.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a M_{W} greater than about 3000 and preferably greater than about 10,000 and a M_{W} that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term “primarily” with respect to directional delivery, refers to an amount greater than 50% of the total amount of therapeutic agent provided to a blood vessel.

The term “restenosis” refers to the renarrowing of an artery following an angioplasty procedure which may include stenosis following stent implantation.

The term “substantially linear release profile” refers to a release profile defined by a plot of the cumulative drug released versus the time during which the release takes place in which the linear least squares fit of such a release profile plot has a correlation coefficient value, r², of greater than 0.92 for data time points after the first day of delivery.

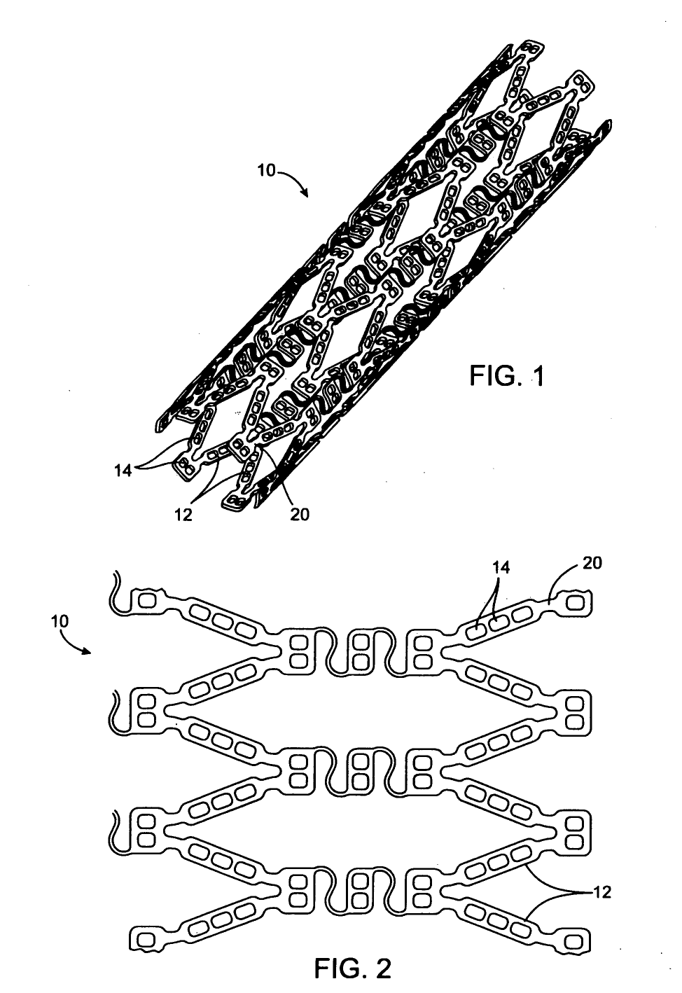

FIG. 1 illustrates one example of an implantable medical device in the form of a stent 10. FIG. 2 is an enlarged flattened view of a portion of the stent of FIG. 1 illustrating one example of a stent structure including struts 12 interconnected by ductile hinges 20. The struts 12 include openings 14 which can be non-deforming openings containing a therapeutic agent. One example of a stent structure having non-deforming openings is shown in U.S. Pat. No. 6,562,065 which is incorporated herein by reference in its entirety.

The implantable medical devices of the present invention are configured to release at least one therapeutic agent from a matrix within a reservoir in the implantable body. The matrix is formed such that the distribution of the agent in the polymer matrix directly controls the rate of elution of the agent from the matrix.

In one embodiment, the matrix is a polymeric material which acts as a binder or carrier to hold the agent in or on the stent and/or modulate the release of the agent from the stent. The polymeric material can be a bioresorbable or a non-bioresorbable material.

The therapeutic agent containing matrix can be disposed within volumes defined by the stent, such as openings, holes, or concave surfaces, as a reservoir of agent, arranged in or on all or a portion of surfaces of the stent structure. When the therapeutic agent matrix is disposed within openings in the structure of the stent to form a reservoir, the openings may be partially or completely filled with matrix containing the therapeutic agent.

Pimecrolimus is a lipophilic anti-inflammatory agent available from Novartis and is currently used in the form of a cream. Pimecrolimus is a calcineurin inhibitor which binds to FKBP-12 and inhibits calcium dependent phosphatase (calcineurin).

Pimecrolimus, an ascomycin macrolactam derivative, is a potent inhibitor of secretion and/or release of multiple inflammatory cytokines from T lymphocytes, mast cells and neutrophils. Via direct effects on lymphocytes, neutrophils, and mast cells and indirect effects on macrophages, Pimecrolimus can modify stent-associated inflammatory events. Pimecrolimus exhibits high activity in models of skin inflammation, but has only very low activity in models of systemic immunosuppression.

Pimecrolimus was developed and launched by Novartis in 2002 for the potential treatment of psoriasis and allergic, irritant and atopic dermatitis. The interest in Pimecrolimus has been substantial due to its significant anti-inflammatory activity and immunomodulatory capabilities and its low systemic immunosuppressive potential.

Unlike Rapamycin, Pimecrolimus does not act via inactivation of mammalian target of Rapamycin (mTOR) and hence does not affect cell cycle regulation. Pimecrolimus is highly lipophilic and crosses cell membranes to bind with high affinity to cellular FK binding protein 12 (FKBP-12). This complex which is distinct from the FKBP-12-TOR complex, interferes with calcineurin, calmodulin receptor related pathways, and leads to inhibition of T cell, mast cell and neutrophil activation by blocking the transcription of early cytokines. In particular it inhibits the production of IL-2, IL-4, tumor necrosis factor α and interferon gamma.

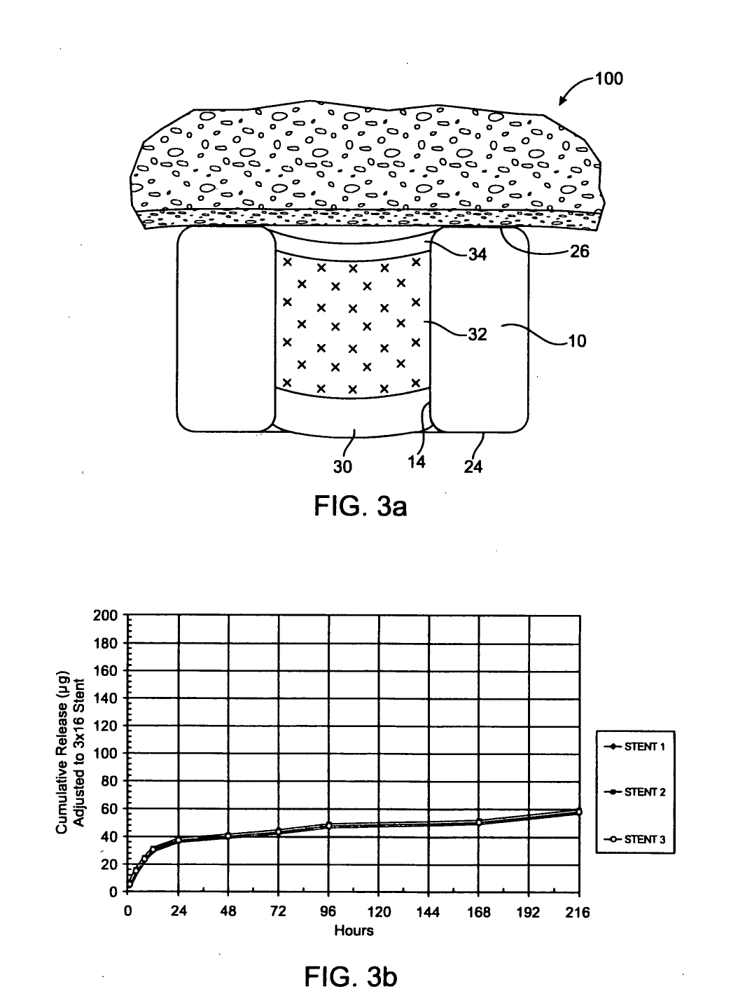

FIG. 3a is a cross section of one strut of the stent 10 and blood vessel 100 illustrating one example of an opening 14 arranged adjacent the vessel wall with a mural surface 26 abutting the vessel wall and a luminal surface 24 opposite the mural surface. The opening 14 of FIG. 3a contains an inlay formed of three regions, a base region 30, a therapeutic agent region 32, and a cap region 34. The therapeutic agent is illustrated as Xs in the matrix. The luminal side 24 of the stent opening 14 is provided with the base region 30. The base region 30 acts as a barrier and causes the therapeutic agent to be delivered primarily to the mural side 26 of the stent.

The matrix and therapeutic agent are arranged in a programmable manner to achieve a desire release rate and administration period which will be described in further detail below. This configuration in which the drug can be precisely arranged within the matrix allows the release rate and administration period to be selected and programmed to a particular application. The methods by which the drug can be precisely arranged within the matrix in the openings is a stepwise deposition process is further described in U.S. patent application Ser. No. 10/777,283 filed on Feb. 11, 2004, which is incorporated herein by reference.

FIGS. 3a-5a illustrate different arrangements of an immunosuppressant or anti-inflammatory agent in reservoirs. Numerous other useful arrangements of the matrix and therapeutic agent can be formed to achieve the release described herein. Each of the areas of the matrix may include one or more agents in the same or different proportions from one area to the next. The matrix may be solid, porous, or filled with other drugs or excipients. The agents may be homogeneously disposed or heterogeneously disposed in different areas of the matrix. Although the various regions shown in FIGS. 3a-5a are shown as distinct regions with definitive tide lines therebetween, it should be understood that depending on manufacturing procedures, the margins of these regions may be mixed together resulting in a continuously varying matrix configuration. In addition, the regions have been shown as slightly concave which is the result of surface tension and may occur in greater or lesser amounts depending on the materials of the matrix and the agent involved.

### EXAMPLE 1

In one example, the stent of FIG. 3a includes a barrier region 30 of PLGA, a therapeutic agent region 32 of PLGA and Pimecrolimus at a drug/polymer ratio of 1/1, and a cap region 34 of PLGA. Each reservoir is filled, by volume, with about 25% barrier, 60% drug/polymer, and 5% cap. The total Pimecrolimus loaded on the stent is about 175 μg on a 3 mm×16 mm stent. The resulting in vitro release is given in FIG. 3b. In this example, the entire release is expected to be completed within about 30 days based on the selection of the polymer.

FIG. 3b shows that after 24 hours, the release profile is substantially linear.

The measurement of in vitro Pimecrolimus release from a stent can be performed according to the following procedure. The in vitro release from other implantable medical devices can be performed in a similar manner by measurement of total drug load and release kinetics by high pressure liquid chromatography (HPLC).

The release kinetics time points are analyzed by reverse phase HPLC. The stents are placed in a release medium of 40% Propylene glycol and 60% Sodium Acetate Buffer (v/v) pH 5 and removed at a plurality of time points. The amount of Pimecrolimus in the release medium samples is determined by HPLC. The following conditions are used:

Analysis Column: Merck Chromolith RPE-18, 3 μm (100 mm×4.6 mm)

Mobile phase:

| | Mobile Phase A | Mobile Phase B (Water with |
|---|---|---|
| Time | (Acetonitrile) | 005% O-phosphoric acid) |
| 0 | 40 | 60 |
| 15 | 70 | 30 |
| 20 | 95 | 5 |
| 25 | 95 | 5 |
| 27 | 40 | 60 |
| 35 | 40 | 60 |

Flow Rate: 1.5 mL/min

Oven Temperature: 60° C.

Detection wavelength: 210 nm

Injection volume: 20 μL

Run time: 35 minutes

Retention time: 16.63 minutes

### EXAMPLE 2

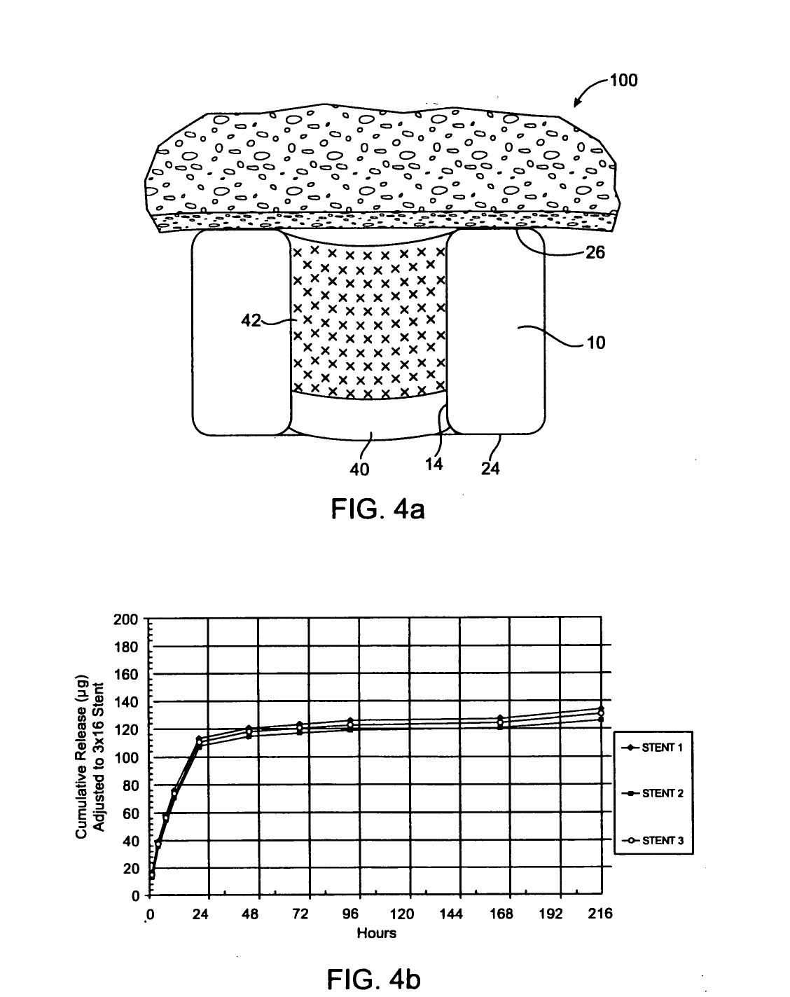

In another example, the stent of FIG. 4a includes a base region 40 of PLGA, and a therapeutic agent region 42 of PLGA and Pimecrolimus at a drug/polymer ratio of 3/1, and no cap region. Each reservoir is filled, by volume, with about 25% base and about 75% drug/polymer. The total Pimecrolimus loaded on the stent is about 300 μg. The resulting in vitro release is given in FIG. 4b. FIG. 4b shows that release is substantially linear after the first 24 hours. About 25 to 50 percent of the drug on the stent is released in the first 24 hours. The entire release is expected to be completed within about 30 days based on the selection of the polymer.

### EXAMPLE 3

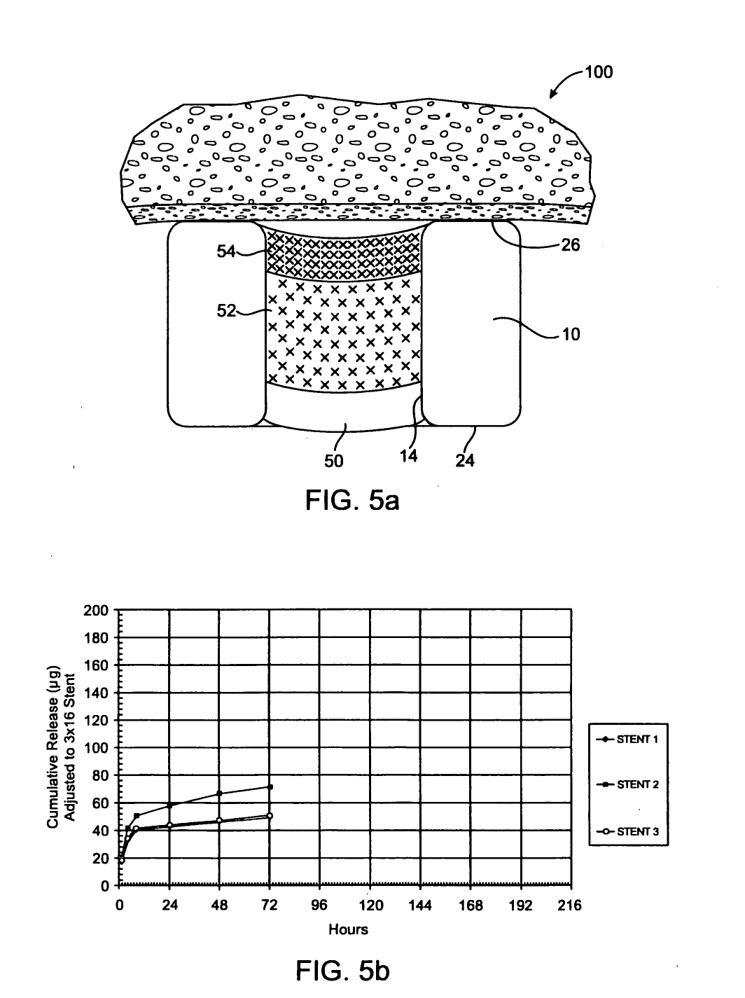

In another example, the stent of FIG. 5a includes a base region 50 of PLGA, and a first therapeutic agent region 52 of PLGA and Pimecrolimus at a drug/polymer ratio of 3/1, a second therapeutic agent region 54 of PLGA and Pimecrolimus at a drug/polymer ratio of 19/1, and no cap region. The total Pimecrolimus loaded on the stent is about 315 μg. Each reservoir is filled, by volume, with about 25% base, 50% first drug/polymer region, and 25% second drug/polymer region. The resulting in vitro release is given in FIG. 5b. FIG. 5b shows that the release is substantially linear after the first 24 hours. About 10 to 25 percent of the drug is released in the first 24 hours. The entire release is expected to be completed within about 60 days based on the selection of the polymer.

### EXAMPLE 4

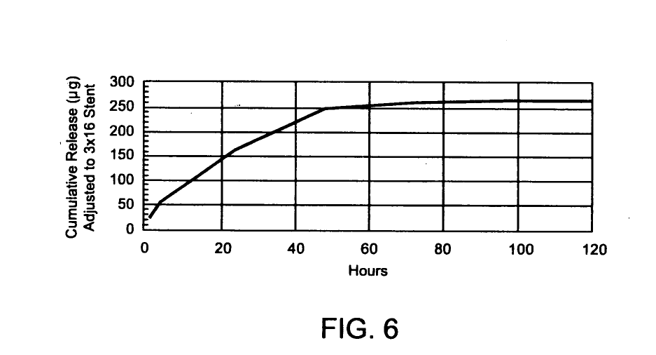

In another example, the stent of FIG. 5a includes a base region 50 of PLGA, and a first therapeutic agent region 52 of about 75% Pimecrolimus and about 25% PLGA, a second therapeutic agent region 54 of about 95% Pimecrolimus and about 5% PLGA, and no cap region. The total Pimecrolimus loaded on the stent is about 325 μg. The reservoirs are filled, by volume, with about 20-25% base, 40-45% first drug/polymer region, and 25-30% second drug/polymer region. The resulting release is given in FIG. 6. FIG. 6 shows and in vitro release of at least about 50% in the first 48 hours followed by a slower linear release.

The above release rates and drug loads have been given for a standard stent of dimensions 3.0 mm in expanded diameter by 16 mm in length. Stents of other dimensions are envisioned to contain total drug loadings in similar respective proportions based on similar drug loading density. The polymer PLGA has been used as one example of a bioresorable polymer, however other polymers and combinations of polymers can also be used. The polymers can also be varied between the regions to further tailor the release. Particularly, the polymer in the base region can be selected to have a higher molecular weight and thus, a slower degradation than the polymer in the other regions to provide drug delivery primarily to the mural side of the stent. In general, the base fills about 10-30% of the volume of the reservoirs, the first drug/polymer fills about 50-80% of the volume of the reservoirs, and the second drug/polymer or cap fills about 0-20% of the volume of the reservoirs.

Therapeutic Agents

The present invention relates to the delivery of immunosuppressant and anti-inflammatory agents including Pimecrolimus. Anti-inflammatory agents include, without limitation, Pimecrolimus salicylic acid derivatives (e.g., aspirin, sodium salicylate, choline magnesium trisalicylate, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast. These anti-inflammatory agents can be delivered alone or in combination. The therapeutic agents for use with the present invention which may be transmitted primarily luminally, primarily murally, or both.

Other therapeutic agents for use with the present invention may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, AZ blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, vasodilators, PPAR gamma agonists, and other miscellaneous compounds.

Antiproliferatives include, without limitation, paclitaxel, actinomycin D, rapamycin, everolimus, tacrolimus, Zotarolimus, cyclosporin, and Pimecrolimus.

Antithrombins include, without limitation, heparin, aspirin, sulfinpyrazone, ticlopidine, ABCIXIMAB, eptifibatide, tirofiban HCL, coumarines, plasminogen, α₂-antiplasmin, streptokinase, urokinase, bivalirudin, tissue plasminogen activator (t-PA), hirudins, hirulogs, argatroban, hydroxychloroquin, BL-3459, pyridinolcarbamate, Angiomax, and dipridamole.

Immunosuppressants include, without limitation, cyclosporine, rapamycin, tacrolimus (FK-506), everolimus, Zotarolimus, etoposide, Pimecrolimus and mitoxantrone.

Antilipid agents include, without limitation, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Antineoplastics include, without limitation, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2′,2′-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, without limitation, insulin, dipyridamole, tirofiban, eptifibatide, abciximab, and ticlopidine.

Angiogenic agents include, without limitation, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydoxyacetone-pyruvate esters and monobutyrin.

Anti-angiogenic agents include, without limitation, endostatin, angiostatin, fumagillin and ovalicin.

Vitamins include, without limitation, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, without limitation, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, without limitation, TIMP-1, TIMP-2, TIMP-3, and SmaPI.

NO donors include, without limitation, L-arginine, amyl nitrite, glyceryl trinitrate, sodium nitroprusside, molsidomine, diazeniumdiolates, S-nitrosothiols, and mesoionic oxatriazole derivatives.

NO release stimulators include, without limitation, adenosine.

Anti-sclerosing agents include, without limitation, collagenases and halofuginone.

Vasoactive agents include, without limitation, nitric oxide, adenosine, nitroglycerine, sodium nitroprusside, hydralazine, phentolamine, methoxamine, metaraminol, ephedrine, trapadil, dipyridamnole, vasoactive intestinal polypeptides (VIP), arginine, and vasopressin.

Endothelial growth factors include, without limitation, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1).

Beta blockers include, without limitation, propranolol, nadolol, timolol, pindolol, labetalol, metoprolol, atenolol, esmolol, and acebutolol.

Hormones include, without limitation, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, without limitation, mevastatin, lovastatin, simvastatin, pravastatin, atorvastatin, and fluvastatin.

Insulin growth factors include, without limitation, IGF-1 and IGF-2.

Antioxidants include, without limitation, vitamin A, carotenoids and vitamin E.

Membrane stabilizing agents include, without limitation, certain beta blockers such as propranolol, acebutolol, labetalol, oxprenolol, pindolol and alprenolol.

Calcium antagonists include, without limitation, amlodipine, bepridil, diltiazem, felodipine, isradipine, nicardipine, nifedipine, nimodipine and verapamil.

Retinoids include, without limitation, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, without limitation, NO donors.

Anti-leukocytes include, without limitation, 2-CdA, IL-1 inhibitors, anti-CD116/CD18 monoclonal antibodies, monoclonal antibodies to VCAM, monoclonal antibodies to ICAM, and zinc protoporphyrin.

Cyclooxygenase inhibitors include, without limitation, Cox-1 inhibitors and Cox-2 inhibitors (e.g., CELEBREX® and VIOXX®).

Immunomodulatory agents include, without limitation, immunosuppressants (see above) and immunostimulants (e.g., levamisole, isoprinosine, Interferon alpha, and Interleukin-2).

ACE inhibitors include, without limitation, benazepril, captopril, enalapril, fosinopril sodium, lisinopril, quinapril, ramipril, spirapril, and 2B3 ACE inhibitors.

Cell sensitizers to insulin include, without limitation, glitazones, P PAR agonists and metformin.

Antisense oligonucleotides include, without limitation, resten-NG.

Antisense oligonucleotides include, without limitation, resten-NG.

Cardio protectants include, without limitation, VIP, pituitary adenylate cyclase-activating peptide (PACAP), apoA-I milano, amlodipine, nicorandil, cilostaxone, and thienopyridine.

Petidose inhibitors include, without limitation, omnipatrilat.

Anti-restenotics include, without limitation, include vincristine, vinblastine, actinomycin, epothilone, paclitaxel, paclitaxel derivatives (e.g., docetaxel), rapamycin, rapamycin derivatives, everolimus, tacrolimus, ZoMaxx, and Pimecrolimus.

PPAR gamma agonists include, without limitation, farglitizar, rosiglitazone, muraglitazar, pioglitazone, troglitazone, and balaglitazone.

Miscellaneous compounds include, without limitation, Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEF and PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

While the invention has been described in detail with reference to the preferred embodiments thereof, it will be apparent to one skilled in the art that various changes and modifications can be made and equivalents employed, without departing from the present invention.

What is claimed is:

1. A stent for reducing restenosis comprising:
an expandable stent; and
a matrix affixed to the stent for delivery of Pimecrolimus to a blood vessel, wherein the bioresorbable matrix includes about 100 μg to about 400 μg of Pimecrolimus normalized for a 3 mm by 16 mm stent.

2. The stent of claim 1, wherein the expandable stent comprises a plurality of reservoirs and the bioresorbable matrix is contained within the reservoirs.

3. The stent of claim 1, wherein the matrix is formulated to release the immunosuppressant agent in a two phase release profile with a first phase releasing at least about 50% of the agent at a fast release rate and a second phase releasing the remaining agent at a slower substantially linear release.

4. The stent of claim 1, wherein the dosage of Pimecrolimus on the stent normalized for a 3 mm by 16 mm stent is about 200 to about 350 μg.

5. The stent of claim 1, wherein the agent is provided in the matrix in a ratio of at least 50% agent to 50% polymer.

6. The stent of claim 1, wherein the agent is provided in the matrix in a ratio of at least 75% agent to 25% polymer.

7. The stent of claim 1, wherein the matrix is biodegradable.

8. The stent of claim 7, wherein the biodegradable matrix is polylactic-co-glycolic acid.

9. A method of reducing restenosis comprising:
providing a drug delivery stent having a dosage of Pimecrolimus provided in a plurality of reservoirs for delivery to an artery, the dosage arranged such that at least 40% of the Pimecrolimus is released from the stent within 48 hours of implantation of the stent in the artery;
implanting the stent within an artery of a patient; and
delivering Pimecrolimus from the stent to the artery such that substantially all the Pimecrolimus is released from the stent within about 3 weeks.

10. A stent for reducing restenosis comprising:
an expandable stent having a plurality of reservoirs;
an anti-inflammatory or immunosuppressive agent provided within the reservoirs in a matrix; and
wherein the matrix is formulated to release the immunosuppressant agent in a two phase release profile with a first phase releasing at least 50% of the agent within a first 48 hours after stent implantation, and a second phase releasing the remaining agent thereafter at a slower release rate.

11. The stent of claim 10, wherein the anti-inflammatory or immunosuppressant agent is Pimecrolimus.

12. The stent of claim 10, wherein the first phase releases at least 50% of the agent within a first 24 hours.

13. The stent of claim 10, wherein the second phase releases the agent with a substantially linear release rate between the second day and the fourteenth day.

14. The stent of claim 10, wherein the matrix is formed of a biodegradable polymer.

15. The stent of claim 11, wherein the dosage of Pimecrolimus on the stent normalized for a 3 mm by 16 mm stent is about 100 to about 400 μg.

16. The stent of claim 11, wherein the dosage of Pimecrolimus on the stent normalized for a 3 mm by 16 mm stent is about 200 to about 350 μg.

17. The stent of claim 10, wherein the agent is provided in the matrix in a ratio of at least 50% agent to 50% polymer.

18. The stent of claim 10, wherein the agent is provided in the matrix in a ratio of at least 75% agent to 25% polymer.

19. The stent of claim 10, wherein the agent is a calcineurin inhibitor.

20. A stent for reducing restenosis comprising:
an expandable stent having a plurality of reservoirs; and
a matrix of Pimecrolimus and bioresorbable polymer provided in the plurality of openings, wherein the matrix is at least 50% Pimecrolimus.

21. The stent of claim 20, wherein the matrix is at least 75% Pimecrolimus.
